(19) United States
(12) Patent Application Publication
(10) Pub. No.: US 2025/0186093 A1
(43) Pub. Date: Jun. 12, 2025

(54) SURGICAL INSTRUMENTS AND METHODS FOR SEPARATING IMPLANTS FROM OTHER COMPONENTS

(71) Applicant: Medos International Sàrl, Le Locle (CH)

(72) Inventors: Eric Biester, Barrington, RI (US); Kevin Yeamans, Providence, RI (US); Michael Sorrenti, Middleboro, MA (US)

(21) Appl. No.: 18/969,887

(22) Filed: Dec. 5, 2024

Related U.S. Application Data
(60) Provisional application No. 63/608,151, filed on Dec. 8, 2023.

(51) Int. Cl. A61B 17/70; A61B 17/00; A61B 90/00

(52) U.S. Cl. CPC A61B 17/7091; A61B 90/03; A61B 2017/00477; A61B 2017/00876; A61B 2090/033

(57) ABSTRACT

Surgical instruments and method to aid in separating an implant from another component at a surgical site are disclosed. An example device can include a body with a proximal-facing surface, a distal-facing surface, as well as opposed first and second lateral ends extending therebetween. The device can further include a first arm extending proximally from the body, the first arm being laterally offset from a center of the body toward the first lateral end of the body. The device can further include a second arm extending distally from the body, the second arm being laterally offset from the first arm toward the second lateral end of the body. The device provides a relatively simple instrument that can be employed to aid in separating a reducer or other component from an implanted bone anchor or other implant.

### CROSS-REFERENCE TO RELATED APPLICATIONS

This application claims the benefit of U.S. Provisional Application No. 63/608,151, entitled “Surgical Instruments and Methods for Separating Implants from Other Components,” filed on Dec. 8, 2023. The entire contents of this application are incorporated by reference herein.

### FIELD

This disclosure relates generally to surgical instruments and methods of use and, more particularly, to surgical instruments that can aid in separating an implant from another component at a surgical site.

### BACKGROUND

Fixation systems can be used in orthopedic surgery or neurosurgery to maintain a desired spatial relationship between multiple bones or bone fragments. For example, in spinal surgery, a spinal fixation system can be implanted into a patient to align and/or fix a desired orientation of one or more vertebrae. A typical spinal fixation system can include bone anchors implanted in the vertebrae and longitudinal rods that are secured to the bone anchors by setscrews or other closure mechanisms. Implanting the fixation system can involve multiple steps, e.g., rod reduction, derotation, and setscrew insertion, among others.

During such procedures, various surgical instruments can be coupled to various implants to perform various aspects of a procedure. For example, rod reducers, extensions, and other components can be selectively coupled to an implanted bone screw to facilitate manipulation of vertebra positioning, introduction of a spinal fixation rod or other element, reduction of such a rod or other element toward the implanted bone screw, introduction of a setscrew to fix such positioning, etc.

In some situations, separation of a surgical instrument or component from an implant can be difficult. For example, rigid anatomy or soft tissue can become lodged against the instrument or component in a manner that prevents its easy separation from the implant. As one more particular example, tissue can interfere with movement of arms on a reducer that are used to selectively latch the reducer on to an implanted bone anchor. As a result, removal of the reducer or other component from the implant can be difficult to perform.

This problem can be especially prevalent when utilizing low profile instruments or components in connection with minimally invasive surgical procedures. Further, prior attempts to address the problem utilize additional instruments to aid in separating the reducer or other component from the implant, but these instruments are often complicated, e.g., having multiple pieces, utilizing threaded connections to the reducer or other component, etc. If this kind of problem arises during a procedure, however, a surgeon or other user desires a quick and simple method for separating the reducer or other component from the implant.

Accordingly, there is a need for improved instruments and methods for separating implants, such as a bone anchor, from other components, such as a rod reducer.

### SUMMARY

The present disclosure addresses the above-noted deficiencies and generally relates to various surgical instruments and methods for use that can be employed to separate implants from other components. Such instruments and methods can be employed to assist in separation spinal rod reducers from implanted bone anchors, though they can also be applied to other instrumentation that couples with bone anchors, such as extensions, etc.

An example device can include a body with a proximal-facing surface, a distal-facing surface, as well as opposed first and second lateral ends extending therebetween. The device can further include a first arm extending proximally from the body, the first arm being laterally offset from a center of the body toward the first lateral end of the body. The device can further include a second arm extending distally from the body, the second arm being laterally offset from the first arm toward the second lateral end of the body. The example device therefore provides a relatively simple instrument that can be employed to aid in separating a reducer or other component from an implanted bone anchor or other implant.

Any of a variety of alternative or additional features can be included and are considered within the scope of the present disclosure. For example, in some embodiments, the first arm can be longer than the second arm.

In certain embodiments, the body can include a first shoulder formed along the proximal-facing surface between the first arm and the center of the body. In some embodiments, the body can include a second shoulder formed along the proximal-facing surface adjacent to the second lateral end. In certain embodiments, the second arm can be positioned at a midpoint between the first and second shoulders.

In some embodiments, the instrument or device can include a handle disposed at a proximal end of the first arm. In certain embodiments, the handle can be configured to modularly couple to a proximal end of the first arm.

In certain embodiments, a proximal portion of the first arm can include at least one flat surface configured for applying torque to the first arm. In some embodiments, the proximal portion of the first arm can include a circumferential groove disposed distal to the at least one flat surface.

In some embodiments, a distal portion of the second arm can include bulbous protrusions extending in a direction that is perpendicular to an axis extending between the opposed first and second lateral ends and perpendicular to a longitudinal axis of the second arm.

In certain embodiments, the body can include a sidewall with a length extending between the proximal-facing surface and the distal-facing surface, the length being configured to limit rotation of the instrument when the instrument is disposed between opposed arms of a second surgical instrument.

In some embodiments, the instrument can include at least one magnet disposed along the proximal-facing surface.

An example method of use can include laterally passing a portion of a first surgical instrument between opposed arms of a second surgical instrument that is coupled to an implant. The method can further include moving the first surgical instrument proximally relative to the second surgical instrument to contact a proximal-facing surface of the first surgical instrument against a distal-facing surface of the second surgical instrument. The method can also include moving a proximal end of the first surgical instrument radially outward relative to a longitudinal axis of the second surgical instrument to pivot the first surgical instrument relative to the second surgical instrument, cause a distal end of the first surgical instrument to contact an inner surface of one of the opposed arms of the second surgical instrument, and urge the arm radially outward relative to a longitudinal axis of the second surgical instrument. The method can further include moving the first surgical instrument and the second surgical instrument proximally relative to the implant.

As with the instruments described above, the methods disclosed herein can include any of a variety of additional or alternative steps that are considered within the scope of the present disclosure. For example, in some embodiments, the method can include positioning the first surgical instrument relative to the second surgical instrument such that opposed first and second shoulders formed along the proximal-facing surface of the first surgical instrument abut outer surfaces of the second surgical instrument.

In certain embodiments, the method can include positioning the first surgical instrument such that a distal-most portion thereof is disposed at a lateral midpoint of a channel formed between the opposed arms of the second instrument.

In some embodiments, the method can include coupling a modular handle to the proximal end of the first surgical instrument.

A variety of implants and instruments can be utilized with the methods disclosed herein. For example, in certain embodiments, the implant can be a bone anchor. In some embodiments, the second surgical instrument can be a spinal rod reducer. In certain embodiments, the method can include removing an inner portion of the spinal rod reducer prior to laterally passing the portion of the first surgical instrument between opposed arms of the spinal rod reducer.

In some embodiments, the proximal end of the first surgical instrument can be part of an arm that is disposed lateral to the second surgical instrument after laterally passing the portion of the first surgical instrument between opposed arms of the second surgical instrument.

In certain embodiments, moving the proximal end of the first surgical instrument radially outward relative to the longitudinal axis of the second surgical instrument can be stopped by contact between the second surgical instrument and a portion of the first surgical instrument that is disposed between the distal end of the first surgical instrument and the proximal-facing surface of the surgical instrument.

In some embodiments, the opposed arms of the second surgical instrument can be pivoting arms configured to latch on to a feature of the implant and the method further comprises squeezing proximal ends of the opposed arms radially inward to urge distal ends of the opposed arms radially outward while moving the proximal end of the first surgical instrument radially outward relative to the longitudinal axis of the second surgical instrument.

In certain embodiments, the method can include separating the first and second surgical instruments after moving the first and second surgical instruments proximally relative to the implant.

Any of the features or variations described herein can be applied to any particular aspect or embodiment of the present disclosure in a number of different combinations. The absence of explicit recitation of any particular combination is due solely to avoiding unnecessary length or repetition.

### BRIEF DESCRIPTION OF THE DRAWINGS

The aspects and embodiments of the present disclosure can be more fully understood from the following detailed description taken in conjunction with the accompanying drawings, in which:

FIG. 1 is a perspective view of one embodiment of a remover instrument according to the present disclosure;

FIG. 2 is an alternative perspective view of the remover instrument of FIG. 1;

FIG. 3 is a bottom view of the remover instrument of FIG. 1;

FIG. 4 is a top view of the remover instrument of FIG. 1;

FIG. 5 is a side view of the remover instrument of FIG. 1;

FIG. 6 is a distal end view of the remover instrument of FIG. 1;

FIG. 7 is a proximal end view of the remover instrument of FIG. 1;

FIG. 8 is an exploded view of the remover instrument of FIG. 1;

FIG. 9 is an alternative exploded view of the remover instrument of FIG. 1;

FIG. 10 is a perspective view of a distal portion of the remover instrument of FIG. 1;

FIG. 11 is a top view of the distal portion of FIG. 10;

FIG. 12 is a side view of the distal portion of FIG. 10;

FIG. 13 is a cross-sectional view of the distal portion of FIG. 10 taken along the plane B-B shown in FIG. 12;

FIG. 14 is a proximal end view of the distal portion of FIG. 10;

FIG. 15 is a cross-sectional view of the distal portion of FIG. 10 taken along the plane A-A shown in FIG. 14;

FIG. 16 is a perspective view of a proximal portion of the remover instrument of FIG. 1;

FIG. 17 is a side view of the proximal portion of FIG. 16;

FIG. 18 is a top view of the proximal portion of FIG. 16;

FIG. 19 is a detail view of the portion of FIG. 18 denoted by circle B;

FIG. 20 is a perspective view of one embodiment of a modular handle coupled to the proximal portion of the remover instrument of FIG. 1;

FIG. 21 is an illustration of the remover instrument of FIG. 1 positioned near one embodiment of a reducer instrument coupled to one embodiment of a bone screw;

FIG. 22 is a perspective view of the reducer instrument of FIG. 21;

FIG. 23 is an exploded view of the reducer instrument of FIG. 21;

FIG. 24 is a perspective view of the reducer instrument of FIG. 21 docked to a bone anchor and reducing a spinal rod;

FIG. 25 illustrates separation of an inner component from an outer component of the reducer instrument of FIG. 21;

FIG. 26 illustrates positioning of the remover instrument of FIG. 1 relative to the reducer instrument and bone screw of FIG. 21;

FIG. 27 illustrates further positioning of the remover instrument of FIG. 1 relative to the reducer instrument and bone screw of FIG. 21;

FIG. 28 illustrates the remover instrument of FIG. 1 ready for use to separate the reducer instrument and the bone screw of FIG. 21;

FIG. 29 illustrates use of the remover instrument of FIG. 1 to separate the reducer instrument and the bone screw of FIG. 21;

FIG. 30 illustrates further use of the remover instrument of FIG. 1 to separate the reducer instrument and bone screw of FIG. 21;

FIG. 31 is a perspective longitudinal cross-sectional view of the reducer instrument of FIG. 1 in use to separate the reducer instrument and bone screw of FIG. 21;

FIG. 32 is a longitudinal cross-sectional view of the reducer instrument of FIG. 1 in use to separate the reducer instrument and bone screw of FIG. 21;

FIG. 33 illustrates separation of the remover instrument of FIG. 1 and the reducer instrument of FIG. 21 relative to the bone screw of FIG. 21; and

FIG. 34 is a perspective view of one embodiment of a remover instrument according to the present disclosure.

### DETAILED DESCRIPTION

Certain example embodiments will now be described to provide an overall understanding of the principles of the structure, function, manufacture, and use of the devices, systems, and methods disclosed herein. One or more examples of these embodiments are illustrated in the accompanying drawings. The devices, systems, and methods specifically described herein and illustrated in the accompanying drawings are non-limiting embodiments. The features illustrated or described in connection with one embodiment may be combined with the features of other embodiments. Such modifications and variations are intended to be included within the scope of the present disclosure.

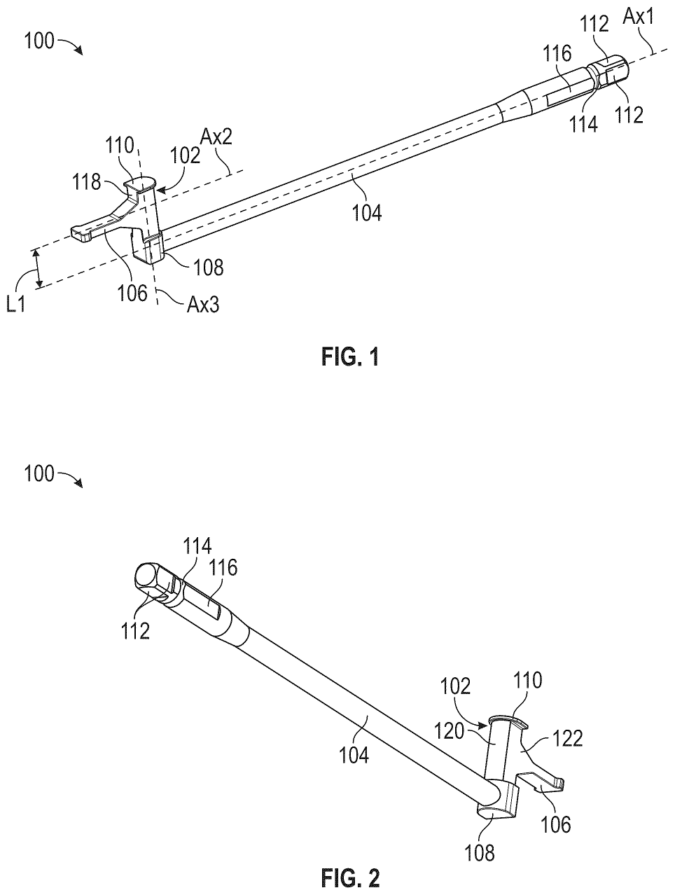

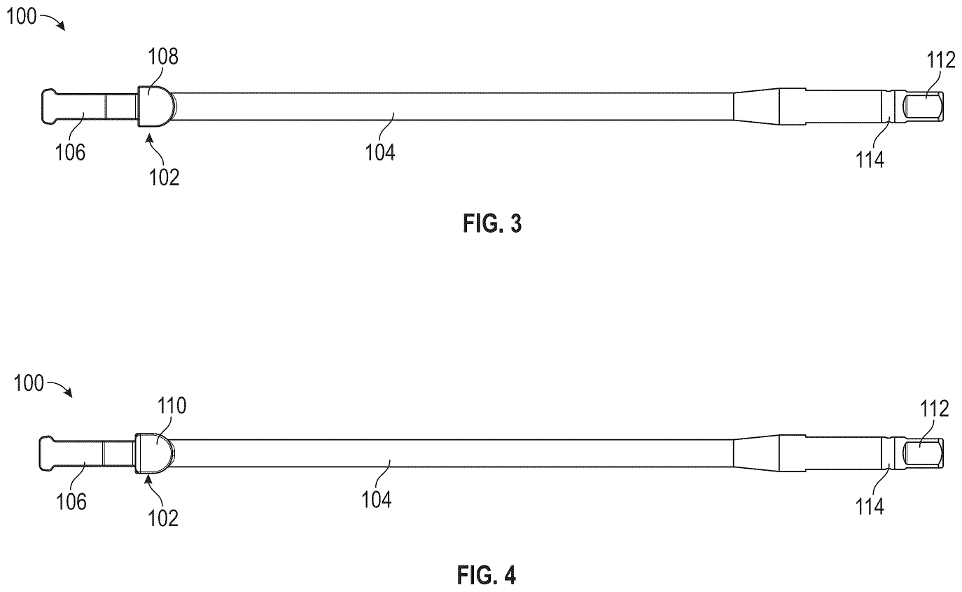

FIGS. 1-7 illustrate various views of one embodiment of a remover instrument 100 according to the present disclosure. The remover instrument 100 can include a body 102, a first arm 104 extending proximally from the body, and a second arm 106 extending distally from the body. As explained below in further detail, the remover instrument 100 can be positioned relative to another surgical instrument or component such that the body 102 contacts a portion of the other surgical instrument and utilizes this as a fulcrum when force is applied by a user to the proximally extending arm 104. Movement of the arm 104 in one direction in response to force exerted thereon by a user can result in opposite movement of the distally extending arm 106 as the instrument 100 rotates about the contact between the fulcrum on the other surgical instrument and the body 102. In some embodiments, the proximally extending arm 104 can have a greater length along its longitudinal axis Ax1 than a length of the distally extending arm 106 along its longitudinal axis Ax2. The mismatch in lengths extending from the body 102 can both accommodate the more constrained space available toward the distal end of the instrument when disposed in proximity to another instrument or component and can allow a user's force to be multiplied according to the mechanical advantage provided by differing lengths of the arms. The high load imparted to the distally extending arm 106 can aid in releasing the other surgical instrument from the implant.

The first and second arms 104, 106 can be laterally offset from one another. That is, the longitudinal axes Ax1, Ax2 of each arm 104, 106 can extend parallel to one another but separated by a length or distance L1 extending along an axis Ax3 that extends along the body 102 and is perpendicular to the longitudinal axes Ax1, Ax2. In particular, the first arm 104 can be laterally offset from a center of the body 102 toward a first lateral end 108 of the body 102. The second arm 106 can be laterally offset from the first arm toward a second lateral end 110 of the body 102.

A proximal portion of the first arm 104 can include one or more features configured to facilitate coupling to a modular handle or otherwise interfacing with a user or other instrument to facilitate transmission of forces therebetween. For example, the proximal portion of the first arm 104 can include at least one flat surface 112 configured for applying torque to the first arm. In the illustrated embodiment, a series of four flat surfaces 112 are disposed around a circumference of a proximal portion of the first arm 104. In addition, a proximal portion of the first arm 104 can include a circumferential groove 114 disposed distal to the at least one flat surface 112, which can be utilized to facilitate coupling with a modular handle, as described in more detail below. Further, one or more flat surfaces 116 can also extend further distally along the first arm 104 to provide additional interfaces for applying force to the first arm or coupling additional components to the arm 104. While in the illustrated embodiment the first arm 104 has a generally cylindrical shape with flat surfaces formed at a proximal end thereof, it is possible in other embodiments that the first arm 104 can have other shapes, including those with a cross-sectional shape that is triangular, rectangular, pentagonal, octagonal, or other shape, such that the arm includes one or more flat sides extending substantially its entire length.

The body 102 of the remover instrument 100 can be a transversely extending bar with a distal-facing surface 118 and a proximal-facing surface 120. The lateral ends 108, 110 can extend between the two surfaces 118, 120. Further, the body 102 can include one or more sidewalls 122 extending between the two lateral ends 108, 110 and between the distal-facing and proximal-facing surfaces 118, 120. The one or more sidewalls 122 can define a dimension L2 extending between the distal-facing and proximal-facing surfaces 118, 120. As explained in more detail below, the dimension L2 can be selected to limit a degree of pivoting or rotation of the remover instrument 100 when in use with another surgical instrument or component so as to prevent damage to the other surgical instrument or component.

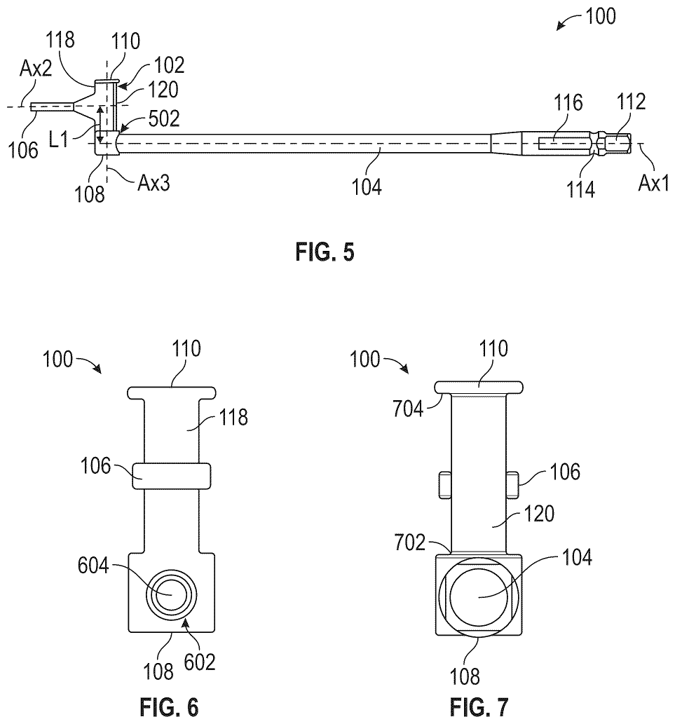

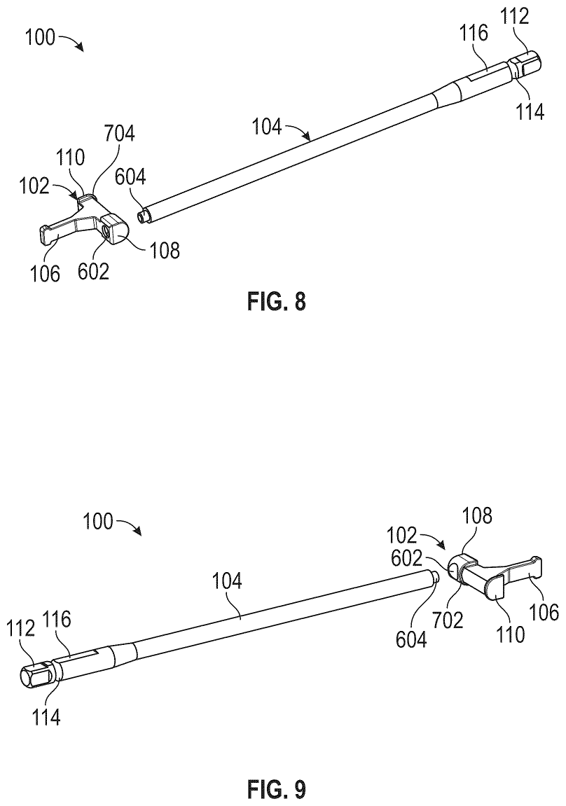

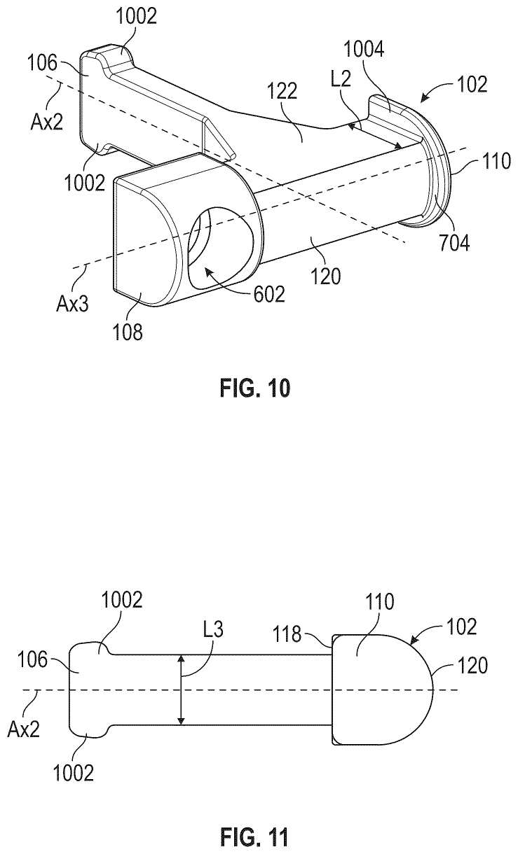

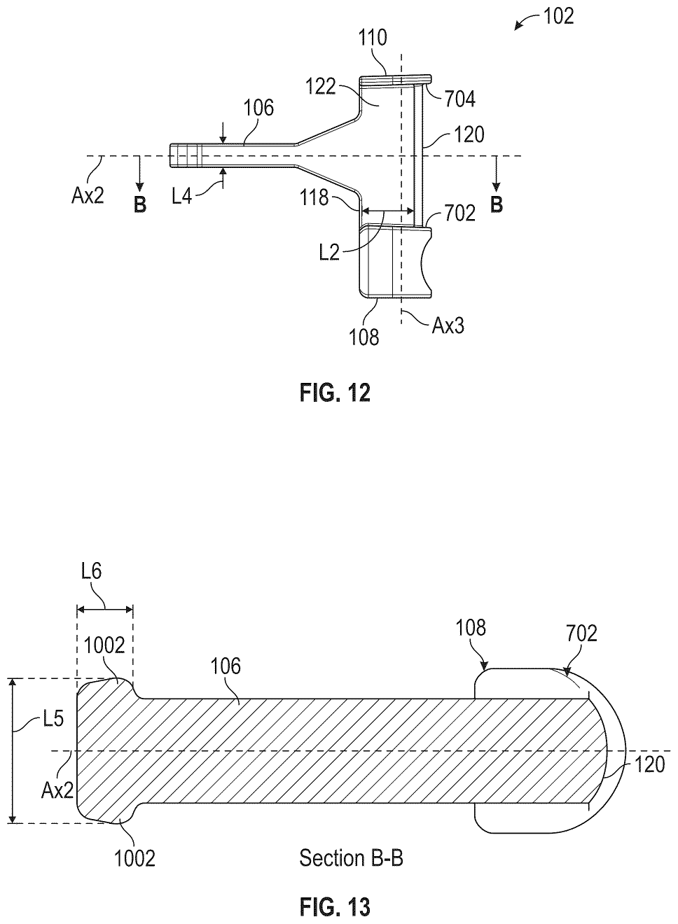

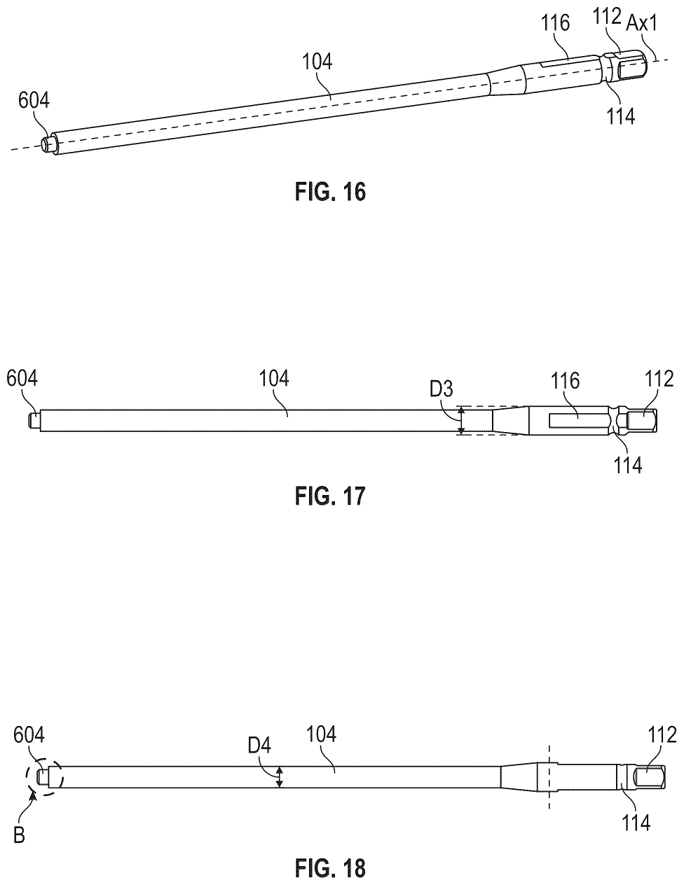

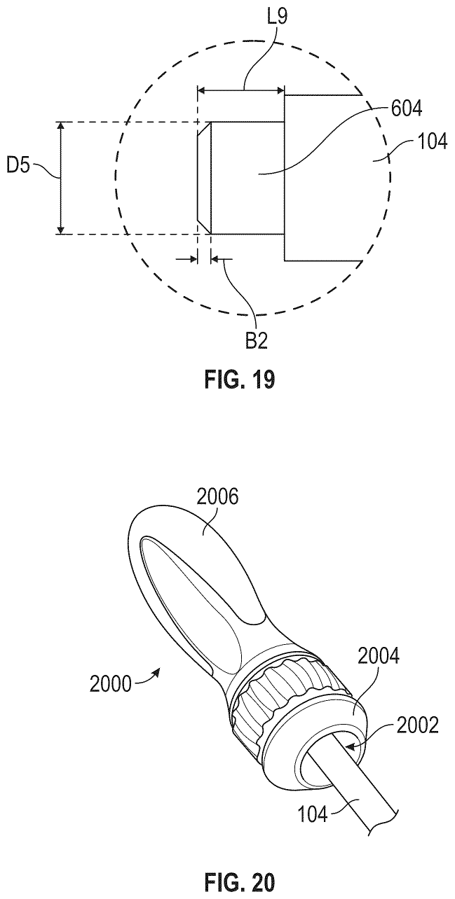

In some embodiments, the remover instrument 100 can be unitary and formed from a single continuous piece of material. In other embodiments, the remover instrument 100 can be formed from a plurality of components coupled to one another in a variety of manners that may or may not facilitate selective disassembly. In the illustrated embodiment, for example, the remover instrument 100 is formed from two components: a first component consisting of the body 102 and distally extending arm 106, as well as a second component consisting of the proximally extending arm 104. FIGS. 8 and 9 illustrate an exploded view of these two components, while FIGS. 10-15 illustrate the first component in more detail and FIGS. 16-19 illustrate the second component in more detail. The components can be joined by a weld 502, as shown in FIG. 5, though other coupling interfaces (or combinations thereof) are possible, including, for example, a threaded coupling, an interference fit, adhesive coupling, etc. The distal-end view of FIG. 6 illustrates a through hole 602 that can be formed in the body 102 toward the lateral end 108 to receive a distal portion 604 of the proximally extending arm 104.

The body 102 can include one or more shoulders formed adjacent to its lateral ends 108, 110. For example, FIG. 7 illustrates a first shoulder 702 formed adjacent the first lateral end 108 and a second shoulder 704 formed adjacent the second lateral end 110. The shoulders 702, 704 can be enlarged portions or wings formed along lateral portions of the body 102. The shoulders 702, 704 can extend along the proximal-facing surface 120 of the body. As shown in FIG. 10 and highlighted by portion 1004, the shoulders 702, 704 can also extend beyond the proximal-facing surface 120 and along the sidewall 122 towards the distal-facing surface 118 of the body 102. As explained in more detail below, the shoulders 702, 704 can serve as guides to aid in positioning the remover instrument 100 relative to another surgical instrument or component, e.g., the shoulders 702, 704 can abut opposed outer surfaces of another surgical instrument or component to center it therebetween and prevent inadvertent lateral movement or misalignment between the two components. Further, the distally extending arm 106 can be positioned at a midpoint between the first and second shoulders, such that it can be positioned near a center or midpoint of another surgical instrument or component when the shoulders 702, 704 are used as guides for positioning the remover instrument 100 relative to the other surgical instrument or component.

As shown in FIGS. 10, 11, and 13, a distal portion of the second, distally extending arm 106 can include one or more bulbous protrusions 1002 extending in a direction that is perpendicular to the longitudinal axis Ax2 of the arm 106 and perpendicular to the axis Ax3 that extends between the opposed first and second lateral ends 108, 110 of the body 102. In the illustrated embodiment, symmetric protrusions 1002 are formed on opposed sides of the arm 106, which can allow the remover instrument 100 to be pivoted in either direction without preference, whereas in other embodiments a protrusion can be included on only one side of the arm 106 and may provide the instrument 100 with a favored pivoting direction.

The distally extending arm 106 can have a dimension L3 extending perpendicular to the plane formed by the axes Ax2 and Ax3, as well as a dimension L4 extending along axis Ax3. The one or more protrusions 1002 can extend outward from the arm 106 along a distal portion thereof to define a dimension L5 that is greater than the dimension L3. The one or more protrusions 1002 can also extend along a distal portion of the arm 106 to define a dimension L6 extending along the axis Ax2. The one or more protrusions 1002 can have a variety of shapes and the various dimensions L3-L6 can vary according to the size of the remover instrument 100, the size of instrument or component the remover instrument is utilized with, etc.

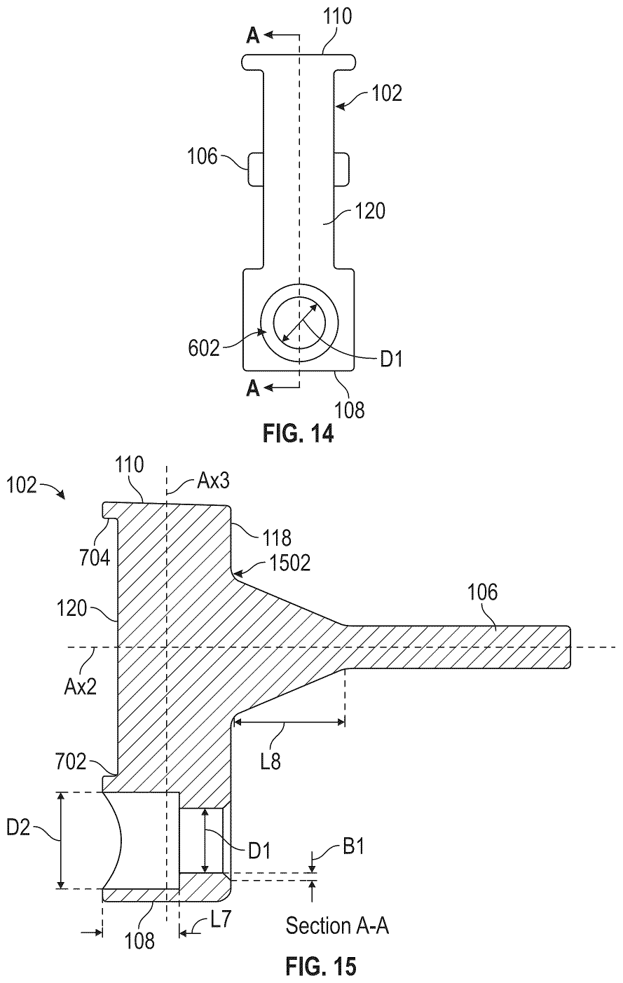

FIGS. 14 and 15 illustrate, among other things, the geometry of the through hole 602 in greater detail. The through hole 602 can include a first diameter D1 extending throughout the body 102 and, more particularly, a portion thereof near the first lateral end 108. A proximal portion of the through hole 602 can have a second diameter D2 that is larger than diameter D1 and can extend a dimension L7 from a proximal end of the body toward a distal end of the body, i.e., along the axis Ax2. The transition between the two diameters can form a step with perpendicular surfaces, as shown in FIG. 15, or can include sloped or ramped transitions between the two diameters. The configuration of varying diameters can be complementary to the profile of a distal portion of the proximally extending arm 102, as described in more detail below and shown in FIGS. 16-19. In addition, a distal portion of the through hole 602 can include a bevel B1. As noted above, the proximally extending arm 104 can be coupled to the body 102 in a variety of manners, some of which can facilitate selective disassembly and some of which are more permanent.

FIG. 15 also illustrates that a proximal portion 1502 of the distally extending arm 106 extending a dimension L8 along the axis Ax2 can have an increasing thickness or dimension along the axis Ax3 moving proximally toward the body 102. This transition region can aid in providing the arm 106 with desired strength and/or rigidity to support the forces it experiences during use.

FIGS. 16-19 illustrate the proximally extending arm 104 in greater detail. As noted above, the arm 104 can have a generally cylindrical shape with a circular cross-section along most of its length. The arm 104 can include one or more transitions of diameter along its length. For example, a proximal portion of the arm 104 can be configured to interface with a driver handle or other component and can have a diameter D3 that larger than a diameter D4 of the arm along a more distal portion. The one or more flats 112, circumferential groove 114, and one or more additional flats 116 can be formed along the proximal portion of the arm 104. Further, a distal portion of the arm 104 can include a geometry complementary to the through hole 602 formed in the body 102. For example, the diameter D4 can match the diameter D2 of the proximal portion of the through hole 602. A distal portion of the arm 104 extending along dimension L9 can transition to a diameter D5 that is less than diameter D4 and can match the diameter D1 of the distal portion of the through hole 602. Still further, a distal outer edge of the arm 104 can include a bevel B2 to facilitate insertion of the arm 104 into the through hole 602 of the body 102. As noted above, once the body 102 and arm 104 are positioned relative to one another, they can be secured relative to one another in a variety of manners, including welding, adhesive, threaded coupling, etc.

FIG. 20 illustrates one embodiment of a modular handle 2000 that can be selectively coupled to a proximal portion of the arm 104 during use of the remover instrument 100. The modular handle 2000 is just one embodiment and a variety of alternative handle embodiments are also possible (e.g., the handle could have a generally “T” shape, etc.). The modular handle 2000 can include a distal-facing socket or recess 2002 that can be configured to receive a proximal portion of the arm 104 therein, a lock 2004 configured to selectively secure the handle 2000 to the arm 104, and a proximal grasping portion 2006 configured to interface with a user's hand.

The recess 2002 can have one or more flat surfaces extending along an inner surface thereof that can be configured to abut the one or more flat surfaces 112 and/or 116 formed along a proximal portion of the arm 104. As a result, the modular handle 2000 can be coupled to the arm 104 in a manner that allows the transmission of various forces and the manipulation of the remover instrument 100 by a user grasping the proximal portion 2006 of the modular handle 2000.

The lock 2004 can interface with the circumferential groove 114 formed along a proximal portion of the arm 104 to prevent undesired axial separation of the modular handle 2000 from the arm 104. A variety of mechanisms are possible, including, in one embodiment, the lock 2004 controlling radial movement of one or more balls that can be selectively seated within the circumferential groove 114. For example, the lock 2004 can be biased to a position in which it urges one or more balls radially inward and prevents their movement radially outward within the modular handle 2000. A user can actuate the lock 2004, e.g., by moving it proximally, to release the one or more balls and allow them to move radially outward. The user can advance the handle 2000 over the distal end of the arm 104 while actuating the lock 2004 in this manner and can subsequently release the lock 2004. As the arm 104 continues to move into the recess 2002, the one or more balls can align with the circumferential groove 114 and move radially inward to be seated within the groove. Once this occurs, the modular handle 2000 will not be able to axially separate from the arm 104 until the user actuates the lock 2004 again to allow radially outward movement of the one or more balls.

The modular handle 2000 shown in the figures can be selectively coupled to the arm 104 and provide various potentially desirable functionality, including the ability to disassemble the components for cleaning and sterilization, the use of differently shaped handles, etc. In some embodiments, however, a modular handle may not be needed or desired and can be replaced with a handle that is unitary or integrally formed with the arm 104 and/or remainder of the remover instrument 100.

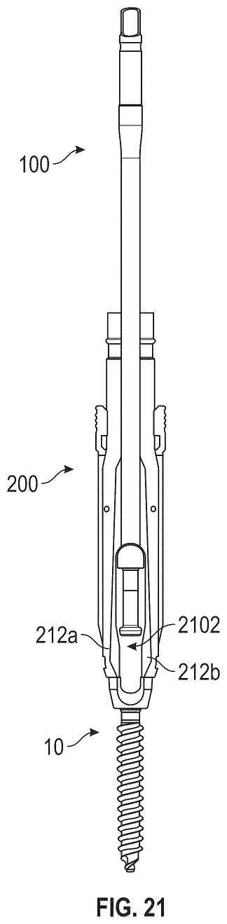

The remover instrument 100 described above can be utilized to assist in separating an implant, such as a bone anchor, from a variety of instrumentation that may be coupled thereto. One example illustrated in FIG. 21 is use of the remover instrument 100 to separate a bone anchor implant 10 from a reducer instrument 200. The reducer 200 can be coupled to the bone anchor 10 that can be implanted in a patient's vertebra. The reducer 200 can be utilized to aid in positioning a spinal fixation rod or other element in a proximal receiver head of the bone anchor 10. As explained below in greater detail, use of the remover instrument 100 can begin with positioning the remover instrument as shown in FIG. 21, i.e., by passing a portion of the remover instrument 100 into a recess or space 2102 between opposed arms 212a, 212b of the reducer 200.

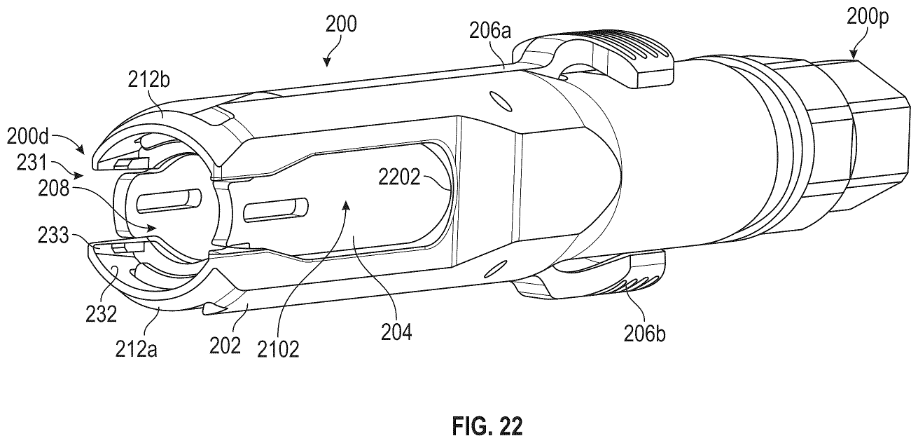

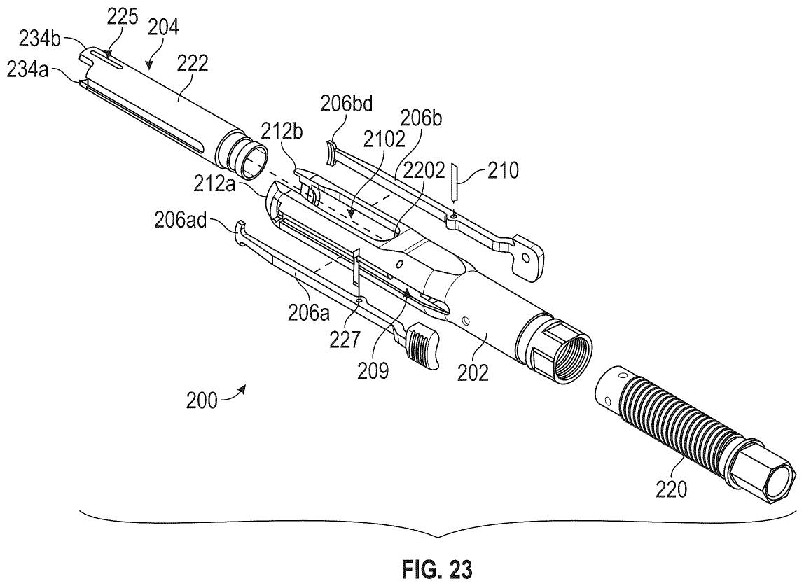

FIGS. 22-24 illustrate the reducer instrument 200 in greater detail. The reducer 200 can include an outer sleeve 202, an inner sleeve 204, and a pair of pivoting arms 206a, 206b attached to the outer sleeve 202. The outer sleeve 202 can define a working channel 208 configured to receive at least a portion of another tool or instrument, e.g., the inner sleeve 204. The channel 208 can provide access to a surgical site to allow passage of instruments or implants therethrough. The channel 208 can extend from a proximal end 200p of the reducer instrument 200 to a distal end 200d of the reducer instrument. The outer sleeve 202 can include a generally tubular central portion that terminates in first and second extensions or arms 212a, 212b. The first and second extensions or arms 212a, 212b can define a recess or space 2102 therebetween and can be joined to one another by a bridge 2202 that can form a proximal end of the recess 2102. The extensions 212a, 212b can include sidewalls 233 extending from an inner surface 232 at the lateral ends of each arm 212a, 212b. The sidewalls 233 can include a tapering profile to aid alignment with a receiver member of the bone anchor 10. The outer sleeve 202 can have a distal pocket 231 formed between opposed arms 212a, 212b that can accept a receiver member of a bone anchor 10 and couple thereto.

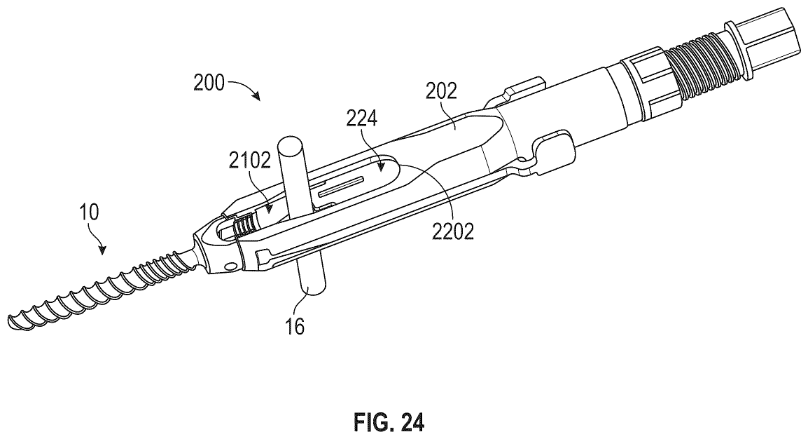

The pivoting arms 206a, 206b can be disposed within opposing recesses 209 formed in the outer sleeve 202. The distal ends 206ad, 206bd of the pivoting arms 206a, 206b and nubs 227 of the pivoting arms 206a, 206b can pass into the channel 208 to interface with the inner sleeve 204 and/or bone anchor 10. Inclusion of the recesses 209 can dispose the pivoting arms 206a, 206b closer to the body of the reducer instrument 200 to narrow the overall profile of the instrument to prevent the arms from interfering with body tissues, other surgical equipment, and the like. The recesses 209 can also provide the windows through which the distally extending arm 106 of the remover instrument 100 can pass to contact the pivoting arms 206a, 206b. Pins 210 can couple the arms 206 to the outer sleeve 202. In use, the reducer instrument 200 can be positioned such that the pivoting arms 206a, 206b engage the bone anchor 10 disposed therebetween to dock the reducer instrument 200 to the bone anchor 10, e.g., as shown in FIG. 24.

The inner sleeve 204 can include a pair of static or fixed arms 234a, 234b for performing rod reduction, as well as a window 225 formed therein to enable observation of a setscrew when threading into the bone anchor during tightening and loosening. As shown, the window 225 can be shaped as a narrow slit that extends along a length of the static arms 234a, 234b to allow for visibility of the setscrew, or another instrument, as it travels through the reducer instrument 200 for engaging the bone anchor.

FIG. 24 illustrates the reducer instrument 200 docked to a bone anchor 10 and reducing a spinal fixation rod 16. The arms 212a, 212b can engage the bone anchor 10, and the spinal rod 16 can be positioned transversely through the outer sleeve 202 between the arms 212a, 212b and distal to the static arms 234a, 234b of the inner sleeve 204 such that distal advancement of the inner sleeve 204 translates the static arms 234a, 234b distally into contact with the spinal rod 16. Further distal advancement translates the inner sleeve 204 through the outer sleeve 202 to force the spinal rod distally towards the bone anchor until the spinal rod is reduced into a rod seat of a receiver member of the bone anchor 10. After reducing the rod 16 into the receiver head of the bone anchor 10, a setscrew can be introduced through the inner sleeve 204 and coupled to the receiver head of the bone anchor to secure the rod relative to the bone anchor.

Additional details regarding the reducer instrument 200 can be found in U.S. Pat. Pub. No. 2022/0280207, entitled “Sequential Reducer.” Additional details regarding the bone anchor 10 can be found in U.S. Pat. Pub. No. 2022/0280200, entitled “Multi-feature Polyaxial Screw.” The entire contents of these publications are incorporated by reference herein.

Following rod reduction and setscrew introduction, the reducer 200 can be separated from the bone anchor 10. In some cases, however, rigid anatomy or soft tissue can become lodged against the bone anchor 10 (or other implant) and/or reducer instrument 200 and/or other component (e.g., if utilizing an extension or other instrument instead of the reducer 200) in a manner that prevents easy separation from the bone anchor 10. As one more particular example, tissue can interfere with movement of pivoting arms 206a, 206b on the reducer 200 that are used to selectively latch the reducer on to the implanted bone anchor. This can happen despite the low-profile design of the reducer 200 and the pivoting arms 206a, 206b that are shaped to avoid soft tissue ingress, etc. As a result, removal of the reducer 200 or other component from the implant 10 can be difficult to perform. In such cases, the remover instrument 100 can be employed to aid in separating the implant 10 from the reducer 200 or other instrument or component.

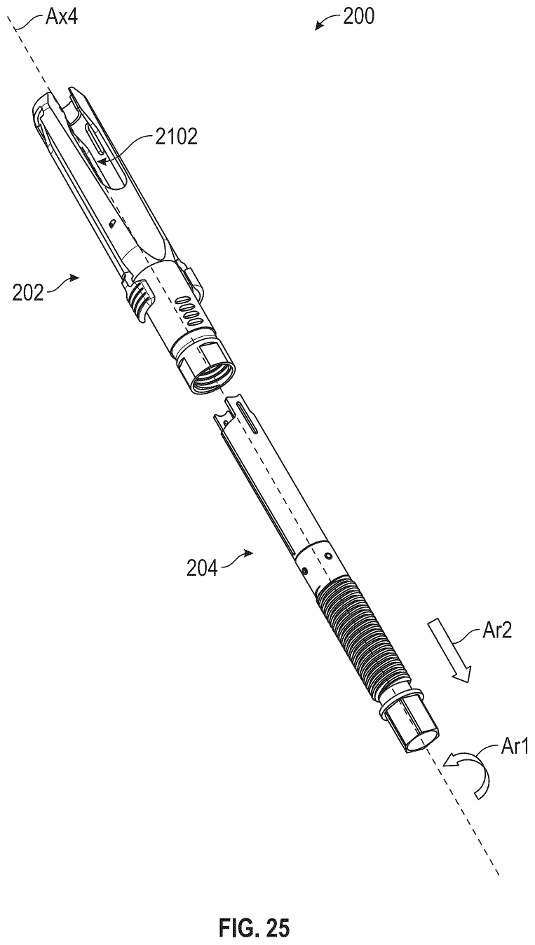

FIGS. 25-33 illustrate one example method of utilizing the remover instrument 100 to aid in separating an implant 10 from a reducer 200 or other surgical instrument or component that may be coupled thereto. The method can include separating the inner sleeve 204 from the outer sleeve 202 of the reducer 200. This can be necessary to free the recess or space 2102 between the arms 212a, 212b of the outer sleeve 202 such that the remover instrument 100 can be passed into the recess 2102, e.g., into the position show in FIG. 21. In contrast, FIG. 22 shows that the inner sleeve 204 can block transverse or lateral access through the recess 2102 of the outer sleeve 202 when the two components of the reducer are assembled.

To separate the inner sleeve 204 from the outer sleeve 202, a user can perform a combination of rotating the inner sleeve 204 relative to the outer sleeve 202 about a longitudinal axis Ax4 of the outer sleeve 202 and proximally withdrawing the inner sleeve 204 relative to the outer sleeve 202 along the axis Ax4, as shown by arrows Ar1 and Ar2 in FIG. 25. These movements can disengage a threaded coupling between the inner sleeve 204 and the outer sleeve 202, and allow the proximal withdrawal of the inner sleeve 204 out of the working channel 208 of the outer sleeve 202.

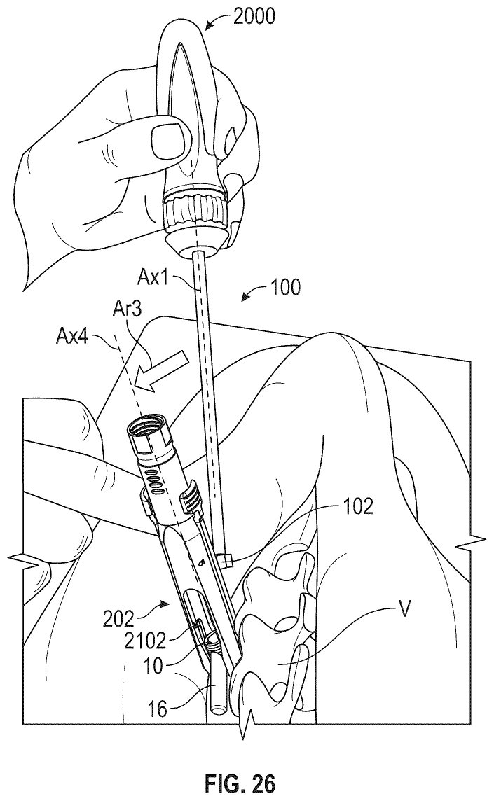

FIG. 26 illustrates a step of laterally or transversely passing a portion of the remover instrument 100 between opposed arms 212a, 212b of the reducer outer sleeve 202 that is coupled to the bone anchor 10 implanted in a patient's vertebra V. More particularly, a user can modularly couple the handle 2000 to the proximally extending arm 104 and utilize the modular handle 2000 to position the remover instrument 100 such that the body 102 and distally extending arm 106 are disposed laterally adjacent to the recess 2102 between the opposed arms 212a, 212b of the reducer outer sleeve 202. The user can move the remover instrument 100 laterally or transversely with respect to the reducer outer sleeve 202 in the direction of arrow Ar3 to pass the body 102 and distally extending arm 106 into the recess 2102.

After passing the body 102 and distally extending arm 106 into the recess 2102, the proximally extending arm 104 can be disposed lateral to the reducer outer sleeve 202 and can extend proximally in a direction that is substantially parallel to a longitudinal axis of the reducer outer sleeve 202. Moreover, the body 102 of the remover instrument 100 can be positioned such that the opposed first and second shoulders 702, 704 formed along the proximal-facing surface 120 abut opposed outer surfaces of the reducer outer sleeve 202. Such positioning can also dispose the distally extending arm 106, including its distal-most portion thereof, at a lateral or transverse midpoint of the recess 2102 between the arms 212a, 212b of the reducer outer sleeve 202.

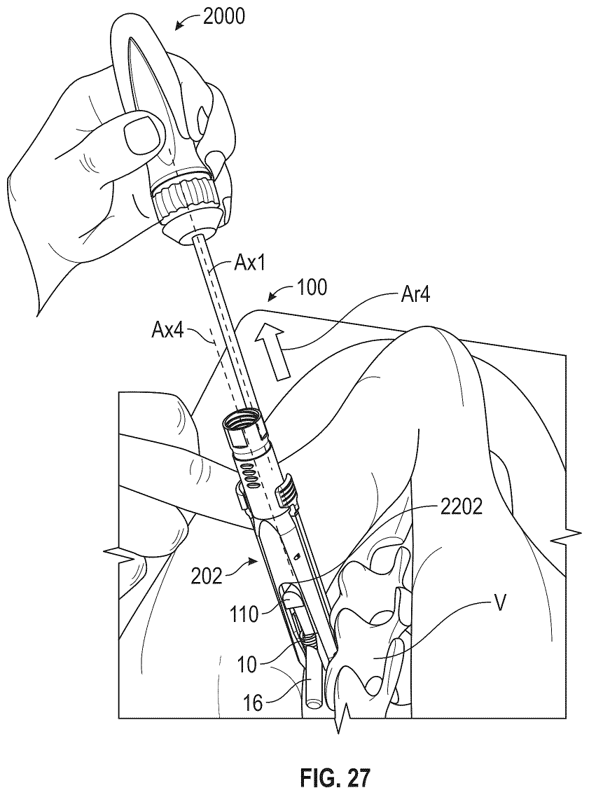

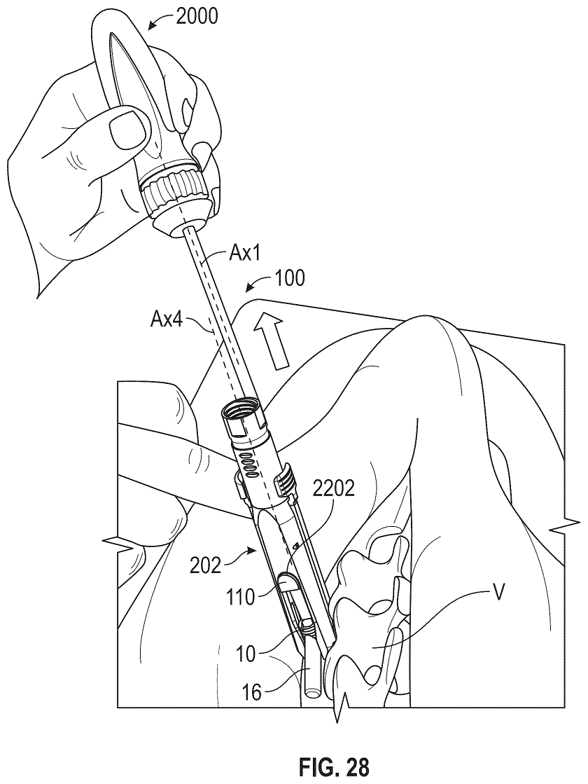

In addition to lateral or transverse positioning of the remover instrument 100 relative to the reducer outer sleeve 202, the method can include moving the remover instrument 100 proximally relative to the reducer outer sleeve 202 to contact the proximal-facing surface 120 of the remover instrument against a distal-facing surface of the reducer outer sleeve 202, e.g., a distal-facing surface of the bridge 2202 extending between the opposed arms 212a, 212b of the reducer outer sleeve 202. This can be accomplished by urging the remover instrument 100 proximally in the direction of arrow Ar4 of FIG. 27 from the position shown in FIG. 27 to the position shown in FIG. 28.

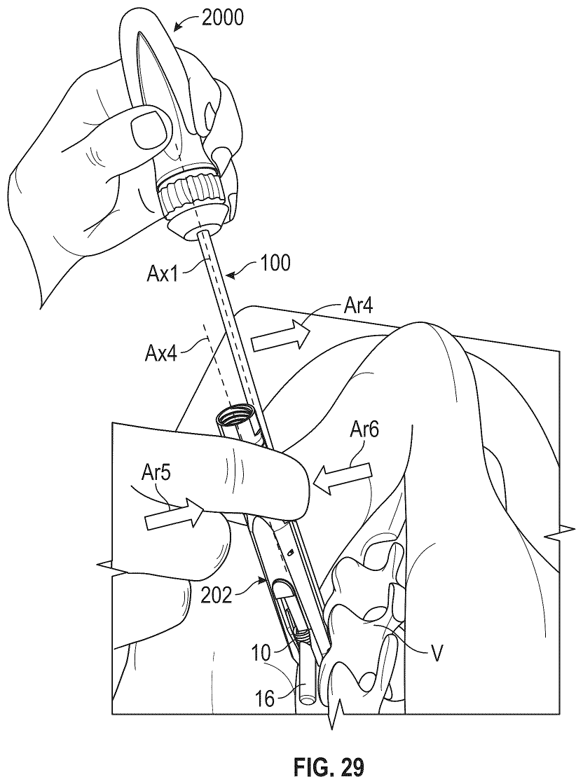

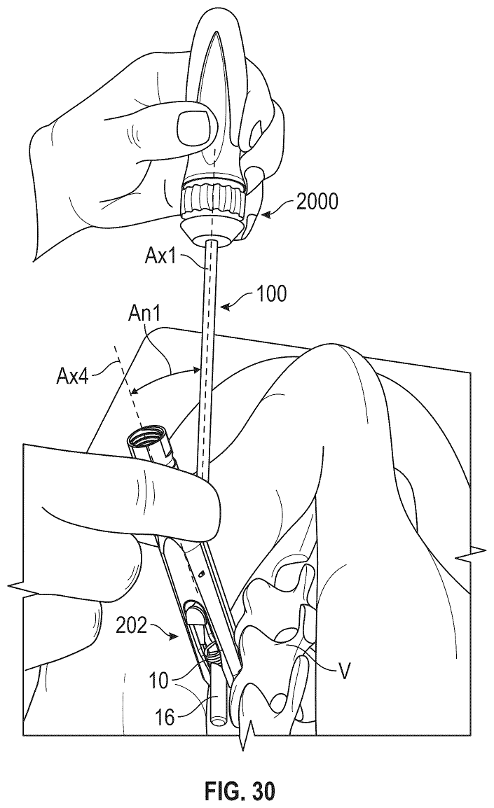

When the remover instrument 100 is positioned such that the proximal-facing surface 120 is in contact with the reducer outer sleeve 202, the instrument 100 is ready for use to aid separation of the reducer outer sleeve 202 from the bone anchor 10. To do so, a user can move the proximally extending arm 104 of the remover instrument 100 radially outward relative to the longitudinal axis Ax4 of the reducer outer sleeve 202. This can pivot the remover instrument 100 relative to the reducer outer sleeve 202 with the reducer serving as a fulcrum for the movement of the remover instrument 100. More particularly, the remover instrument 100 can pivot about the one or more points of contact between the proximal-facing surface 120 and the bridge 2202 of the reducer outer sleeve 202. This motion of the remover instrument 100 relative to the reducer outer sleeve 202 is illustrated by arrow Ar4 in FIG. 29 and the change in position of the remover instrument 100 from FIG. 29 to FIG. 30.

In combination with the above-described movement of the remover instrument 100, a user can also squeeze proximal ends of opposed arms of the reducer outer sleeve 202, e.g., proximal ends of the pivoting arms 206a, 206b, radially inward, as shown by arrows Ar5 and Ar6 in FIG. 29. This can urge distal ends 206ad, 206bd of the opposed arms 206a, 206b radially outward relative to the longitudinal axis Ax4 of the reducer outer sleeve 202. This action is the typical motion utilized to release the reducer outer sleeve 202 from the implant 10 in the absence of the remover instrument 100. The remover instrument 100 is a tool to replace or augment this action from a user. For example, if a user finds that they cannot cause a pivoting arm of the reducer outer sleeve 202 to move by pressing on the proximal end of the pivoting arm alone (e.g., due to interference from soft tissue, hard anatomy, etc.), the remover instrument 100 can be utilized to further urge the distal end of the pivoting arm radially outward to disengage it from the implant 10.

The above-described movement of the remover instrument 100 can cause a distal end of the remover instrument, e.g., a distal portion of the distally extending arm 106, to contact an inner surface of one of the opposed arms of the reducer outer sleeve 202, e.g., a distal portion of one of the pivoting arms 206a, 206b, and urge the arm radially outward relative to the longitudinal axis Ax4 of the reducer outer sleeve 202.

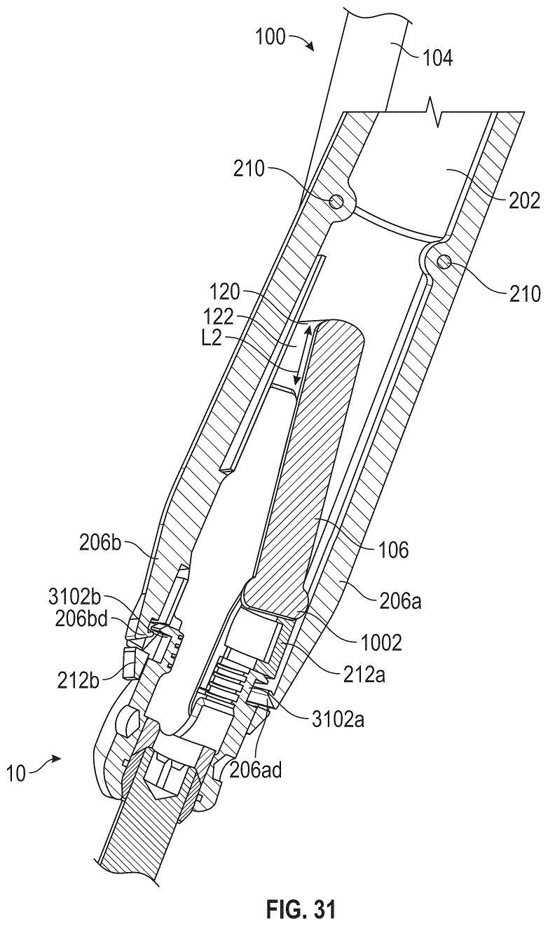

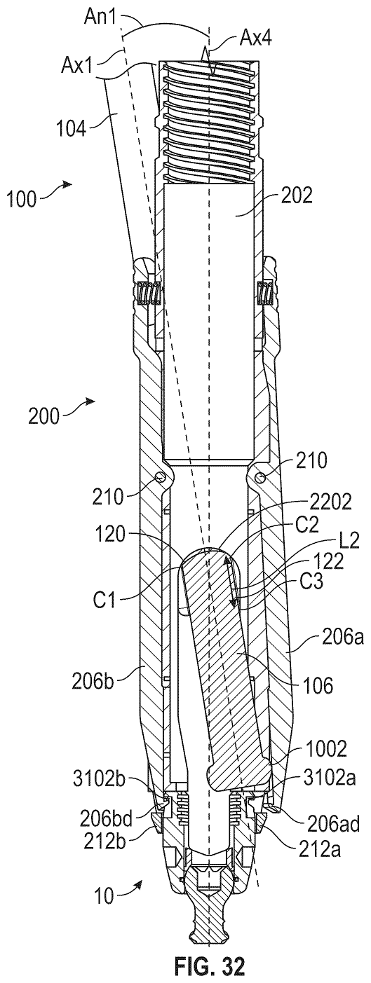

The cross-sectional views of FIGS. 31 and 32 illustrate the operation of the remover instrument 100 in greater detail. As shown in these figures, a distal end 206ad, 206bd of the pivoting arms 206a, 206b of the reducer outer sleeve 202 include hook features that can engage notches or grooves 3102a, 3102b formed around an outer circumference of opposed arms of the receiver head of the bone anchor 10. The engagement of the pivoting arms 206a, 206b with the notches 3102a, 3102b can secure the coupling between the reducer outer sleeve 202 and the bone anchor 10.

As the remover instrument 100 is rotated or moved as described above, it can move from a first position, in which the longitudinal axis Ax1 of the proximally extending arm 104 of the remover instrument is parallel with the longitudinal axis Ax4 of the reducer outer sleeve 202, to a second position, in which the longitudinal axis Ax1 is angled relative to the longitudinal axis Ax4 by an angle An1. Movement of the proximally extending arm 104 in one direction (e.g., to the left in the plane of FIG. 32) can cause opposite movement of the distally extending arm 106 (e.g., to the right in the plane of FIG. 32). As the distally extending arm 106 moves, a distal portion thereof (e.g., the bulbous protrusion 1002) can contact an inner surface of the pivoting arm 206a. Continued movement of the remover instrument 100 can urge the distal end 206ad of the pivoting arm 206a radially outward such that it disengages from the notch 3102a, thereby releasing at least that side of the reducer outer sleeve 202 from the bone anchor 10. Once tension is released in this manner, a user can disengage the other pivoting arm 206b by moving the proximal end of the pivoting arm 206b radially inward. In some cases, a user can tilt the reducer outer sleeve 202 toward the opposite pivoting arm 206b after releasing the pivoting arm 206a with the remover instrument 100 to facilitate release of the pivoting arm 206b.

As noted above, the body 102 of the remover instrument 100 can include a sidewall 122 extending between the distally facing surface 118 and the proximally facing surface 120. The length L2 of the sidewall 122 can be configured to provide a limit on the amount of rotation that is possible between the remover instrument 100 and the reducer outer sleeve 202, i.e., the magnitude of the angle An1. More particularly, the proximally facing surface 120 of the remover instrument 100 can contact a distally facing surface of the reducer outer sleeve 202, e.g., a portion of the bridge 2202. Contact can occur along the proximally facing surface 120 of the remover instrument 100, at least including contact points C1 and C2 at the edges of the surface 120, as shown in FIG. 32. As a user moves the proximally extending arm 104 radially outward relative to the longitudinal axis Ax4, the remover instrument 100 pivots about the body 102 where it contacts the reducer outer sleeve 202. As this motion continues, the body 102 can contact a portion of the reducer outer sleeve 202 at contact point C3 as well. When the body 102 reaches this point, the contact at point C3 will limit further rotation of the remover instrument 100 relative to the reducer outer sleeve 202, thereby capping the angle An1 and the amount of radially outward deflection of the distally extending arm 106. Accordingly, setting the length L2 of the sidewall 122 of the body 102 can limit the angle An1 and amount of radially outward deflection of the arm 106. This can prevent damage to the reducer outer sleeve 202 that might occur if a user could continue to force the distal portion of the pivoting arm 206a radially outward without limit.

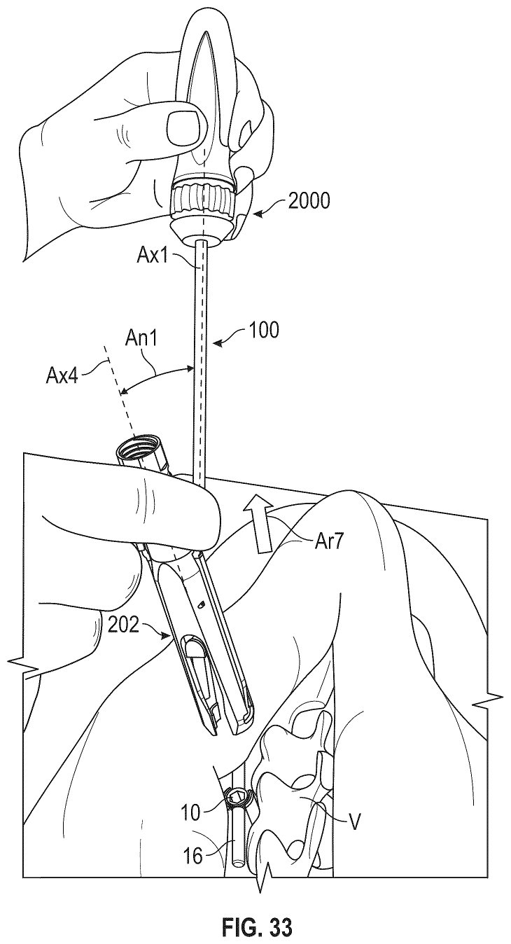

Following release of the reducer outer sleeve 202 from the bone anchor 10, the method can include moving the reducer outer sleeve 202 and the remover instrument 100 proximally relative to the bone anchor 10, thereby separating the bone anchor from the other components. This movement is illustrated in FIG. 33, in particular by arrow Ar7. The remover instrument 100 and reducer outer sleeve 202 can be moved together and a user can later separate these two components from one another, or they can be withdrawn separately.

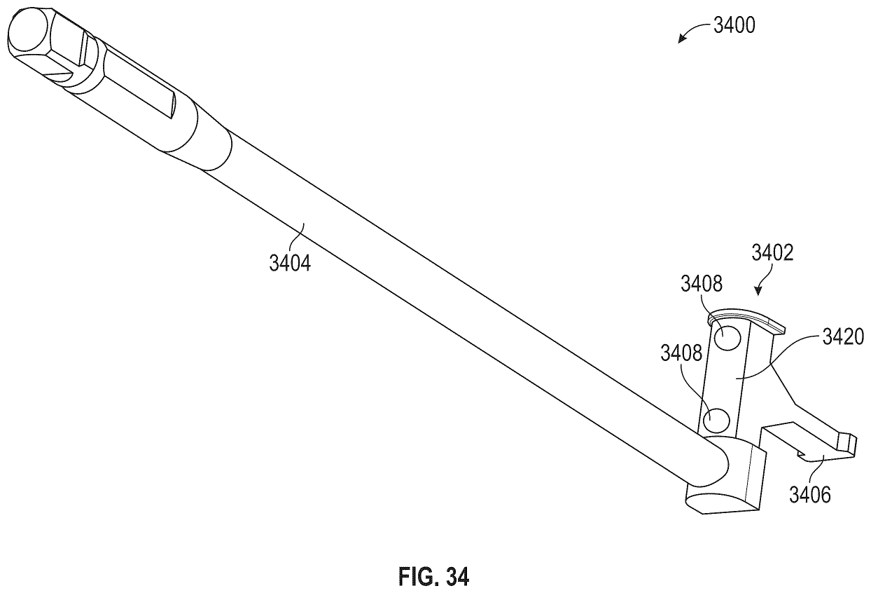

A number of variations and/or additional features are possible and within the scope of the present disclosure. For example, in some embodiments one or more magnets can be incorporated into the remover instrument 100 to aid in positioning the instrument relative to the reducer outer sleeve 202. FIG. 34 illustrates an example remover instrument 3400 that is similar to the instrument 100 in many respects. For example, the remover instrument 3400 includes a body 3402, proximally extending arm 3404, and distally extending arm 3406 that can be similar to the remover instrument 100 described above. In addition, the remover instrument 3400 can include one or more magnets 3408 disposed along the proximal-facing surface 3420 of the body 3402. The one or more magnets 3408 can be disposed along the surface 3420 in a variety of manners, including by affixing magnets to the surface 3420, embedding magnets in one or more recessed formed in the surface, disposing magnets in recesses formed in a distally facing surface such that they are near the opposed proximally facing surface, etc.

The magnets 3408 can be positioned to interact with the distal-facing surface of the reducer outer sleeve 202, e.g., the bridge 2202, and thereby urge the remover instrument 3400 into proper position for use. That is, the magnets 3408 can aid a user in the proximal movement of the instrument 3400 relative to the reducer outer sleeve 202 that is shown by arrow Ar4 in FIG. 27. This proximal movement brings the proximally facing surface 3420 of the body 3402 into contact with the distally facing surface of the bridge 2202 of the reducer outer sleeve 202, which properly establishes the fulcrum that is utilized when actuating the remover instrument 3400 to separate the reducer outer sleeve 202 from the implant 10. The magnets can also aid a user in maintaining this proper relative positioning after initially achieving it, even allowing a user to release the remover instrument 3400 before proceeding to actuate if needed.

Various devices and methods disclosed herein can be used in minimally invasive surgery and/or open surgery. While various devices and methods disclosed herein are generally described in the context of surgery on a human patient, the methods and devices disclosed herein can be used in any of a variety of surgical procedures with any human or animal subject, or in non-surgical procedures.

Various devices disclosed herein can be constructed from any of a variety of known materials. Example materials include those that are suitable for use in surgical applications, including metals such as stainless steel, titanium, titanium nitride, nickel, cobalt, chrome, cobalt-chromium, or alloys and combinations thereof, polymers such as PEEK, ceramics, carbon fiber, and so forth. The various components of the devices disclosed herein can be rigid or flexible. In addition, one or more of the components or devices disclosed herein can be formed as monolithic or unitary structures, e.g., formed from a single continuous material, or can be formed from separate components coupled together in a variety of manners that either facilitate or discourage subsequent separation. One or more components or portions of the device can be formed from a radiopaque material to facilitate visualization under fluoroscopy and other imaging techniques, or from a radiolucent material so as not to interfere with visualization of other structures. Example radiolucent materials include carbon fiber and high-strength polymers. Further, various methods of manufacturing can be utilized, including 3D printing or other additive manufacturing techniques, as well as more conventional manufacturing techniques, including molding, stamping, casting, machining, etc.

Various devices or components disclosed herein can be designed to be disposed of after a single use, or they can be designed to be used multiple times. In either case, however, various devices or components can be reconditioned for reuse after at least one use. Reconditioning can include any combination of the steps of disassembly, followed by cleaning or replacement of particular pieces, and subsequent reassembly. In particular, a device or component can be disassembled, and any number of the particular pieces or parts thereof can be selectively replaced or removed in any combination. Upon cleaning and/or replacement of particular parts, the device or component can be reassembled for subsequent use either at a reconditioning facility, or by a surgical team immediately prior to a surgical procedure. Reconditioning of a device or component can utilize a variety of techniques for disassembly, cleaning/replacement, and reassembly. Use of such techniques, and the resulting reconditioned device or component, are within the scope of the present disclosure.

Various devices or components described herein can be processed before use in a surgical procedure. For example, a new or used device or component can be obtained and, if necessary, cleaned. The device or component can be sterilized. In one sterilization technique, the device or component can be placed in a closed and sealed container, such as a plastic or TYVEK bag. The container and its contents can be placed in a field of radiation that can penetrate the container, such as gamma radiation, x-rays, or high-energy electrons. The radiation can kill bacteria on the device or component and in the container. The sterilized device or component can be stored in the sterile container. The sealed container can keep the device or component sterile until it is opened in the medical facility. Other forms of sterilization are also possible, including beta or other forms of radiation, ethylene oxide, steam, or a liquid bath (e.g., cold soak). Certain forms of sterilization may be better suited to use with different devices or components, or portions thereof, due to the materials utilized, the presence of electrical components, etc.

In this disclosure, articles “a” and “an” are used to refer to one or to more than one (i.e., at least one) of the grammatical object of the article. By way of example, “an element” means at least one element and can include more than one element. The term “about” is used to provide flexibility to a numerical range endpoint by providing that a given value may be “slightly above” or “slightly below” the endpoint without affecting the desired result. The use herein of the terms “including,” “comprising,” or “having,” and variations thereof, is meant to encompass the elements listed thereafter and equivalents thereof, as well as additional elements. As used herein, “and/or” refers to and encompasses any and all possible combinations of one or more of the associated listed items, as well as the lack of combinations where interpreted in the alternative (“or”). Further, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B,” “one or more of A and B,” and “A and/or B” are each intended to mean “A alone, B alone, or A and B together.” A similar interpretation is also intended for lists including three or more items. For example, the phrases “at least one of A, B, and C,” “one or more of A, B, and C,” and “A, B, and/or C” are each intended to mean “A alone, B alone, C alone, A and B together, A and C together, B and C together, or A and B and C together.” In addition, use of the term “based on,” is intended to mean, “based at least in part on,” such that an un-recited feature or element is also permissible.

To the extent that linear, circular, or other dimensions are used in the description of the disclosed devices and methods, such dimensions are not intended to limit the types of shapes that can be used in conjunction with such devices and methods. Equivalents to such dimensions can be determined for different geometric shapes, etc. Further, like-numbered components of the embodiments can generally have similar features. Still further, sizes and shapes of the devices, and the components thereof, can depend at least on the anatomy of the subject in which the devices will be used, the size and shape of objects with which the devices will be used, and the methods and procedures in which the devices will be used.

The figures provided herein are not necessarily to scale. Still further, to the extent arrows are used to describe a direction of movement, these arrows are illustrative and in no way limit the direction that the respective component can or should be moved. Other movements and directions may be possible to create the desired result in view of the present disclosure.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Further features and advantages based on the above-described embodiments are possible and within the scope of the present disclosure. Accordingly, the disclosure is not to be limited by what has been particularly shown and described. All publications and references cited herein are expressly incorporated herein by reference in their entirety, except for any definitions, subject matter disclaimers, or disavowals, and except to the extent that the incorporated material is inconsistent with the express disclosure herein, in which case the language in this disclosure controls.

Examples of the above-described embodiments can include the following:
1. A surgical instrument, comprising:
   a body with a proximal-facing surface, a distal-facing surface, as well as opposed first and second lateral ends extending therebetween;
   a first arm extending proximally from the body, the first arm being laterally offset from a center of the body toward the first lateral end of the body; and
   a second arm extending distally from the body, the second arm being laterally offset from the first arm toward the second lateral end of the body.
2. The instrument of example 1, wherein the first arm is longer than the second arm.
3. The instrument of any of example 1 to 2, wherein the body includes a first shoulder formed along the proximal-facing surface between the first arm and the center of the body.
4. The instrument of any of examples 1 to 3, wherein the body includes a second shoulder formed along the proximal-facing surface adjacent to the second lateral end.
5. The instrument of example 4, wherein the second arm is positioned at a midpoint between the first and second shoulders.
6. The instrument of any of examples 1 to 5, further comprising a handle disposed at a proximal end of the first arm.
7. The instrument of example 6, wherein the handle is configured to modularly couple to a proximal end of the first arm.
8. The instrument of any of examples 1 to 7, wherein a proximal portion of the first arm includes at least one flat surface configured for applying torque to the first arm.
9. The instrument of example 8, wherein the proximal portion of the first arm includes a circumferential groove disposed distal to the at least one flat surface.
10. The instrument of any of examples 1 to 9, wherein a distal portion of the second arm includes bulbous protrusions extending in a direction that is perpendicular to an axis extending between the opposed first and second lateral ends and perpendicular to a longitudinal axis of the second arm.
11. The instrument of any of examples 1 to 10, wherein the body includes a sidewall with a length extending between the proximal-facing surface and the distal-facing surface, the length being configured to limit rotation of the instrument when the instrument is disposed between opposed arms of a second surgical instrument.
12. The instrument of any of examples 1 to 11, further comprising at least one magnet disposed along the proximal-facing surface.
13. A surgical method, comprising:
   laterally passing a portion of a first surgical instrument between opposed arms of a second surgical instrument that is coupled to an implant;
   moving the first surgical instrument proximally relative to the second surgical instrument to contact a proximal-facing surface of the first surgical instrument against a distal-facing surface of the second surgical instrument;
   moving a proximal end of the first surgical instrument radially outward relative to a longitudinal axis of the second surgical instrument to pivot the first surgical instrument relative to the second surgical instrument, cause a distal end of the first surgical instrument to contact an inner surface of one of the opposed arms of the second surgical instrument, and urge the arm radially outward relative to a longitudinal axis of the second surgical instrument; and
   moving the first surgical instrument and the second surgical instrument proximally relative to the implant.
14. The method of example 13, further comprising positioning the first surgical instrument relative to the second surgical instrument such that opposed first and second shoulders formed along the proximal-facing surface of the first surgical instrument abut outer surfaces of the second surgical instrument.
15. The method of any of examples 13 to 14, further comprising positioning the first surgical instrument such that a distal-most portion thereof is disposed at a lateral midpoint of a channel formed between the opposed arms of the second instrument.
16. The method of any of examples 13 to 15, further comprising coupling a modular handle to the proximal end of the first surgical instrument.
17. The method of any of examples 13 to 16, wherein the implant is a bone anchor.
18. The method of any of examples 13 to 17, wherein the second surgical instrument is a spinal rod reducer.
19. The method of example 18, further comprising removing an inner portion of the spinal rod reducer prior to laterally passing the portion of the first surgical instrument between opposed arms of the spinal rod reducer.
20. The method of any of examples 13 to 19, wherein the proximal end of the first surgical instrument is part of an arm that is disposed lateral to the second surgical instrument after laterally passing the portion of the first surgical instrument between opposed arms of the second surgical instrument.
21. The method of any of examples 13 to 20, wherein moving the proximal end of the first surgical instrument radially outward relative to the longitudinal axis of the second surgical instrument is stopped by contact between the second surgical instrument and a portion of the first surgical instrument that is disposed between the distal end of the first surgical instrument and the proximal-facing surface of the surgical instrument.
22. The method of any of examples 13 to 21, wherein the opposed arms of the second surgical instrument are pivoting arms configured to latch on to a feature of the implant and the method further comprises squeezing proximal ends of the opposed arms radially inward to urge distal ends of the opposed arms radially outward while moving the proximal end of the first surgical instrument radially outward relative to the longitudinal axis of the second surgical instrument.
23. The method of any of examples 13 to 22, further comprising separating the first and second surgical instruments after moving the first and second surgical instruments proximally relative to the implant.

What is claimed is:

1. A surgical instrument, comprising:
a body with a proximal-facing surface, a distal-facing surface, as well as opposed first and second lateral ends extending therebetween;
a first arm extending proximally from the body, the first arm being laterally offset from a center of the body toward the first lateral end of the body; and
a second arm extending distally from the body, the second arm being laterally offset from the first arm toward the second lateral end of the body.

2. The instrument of claim 1, wherein the first arm is longer than the second arm.

3. The instrument of claim 1, wherein the body includes a first shoulder formed along the proximal-facing surface between the first arm and the center of the body.

4. The instrument of claim 3, wherein the body includes a second shoulder formed along the proximal-facing surface adjacent to the second lateral end.

5. The instrument of claim 4, wherein the second arm is positioned at a midpoint between the first and second shoulders.

6. The instrument of claim 1, further comprising a handle disposed at a proximal end of the first arm.

7. The instrument of claim 6, wherein the handle is configured to modularly couple to a proximal end of the first arm.

8. The instrument of claim 1, wherein a proximal portion of the first arm includes at least one flat surface configured for applying torque to the first arm.

9. The instrument of claim 8, wherein the proximal portion of the first arm includes a circumferential groove disposed distal to the at least one flat surface.

10. The instrument of claim 1, wherein a distal portion of the second arm includes bulbous protrusions extending in a direction that is perpendicular to an axis extending between the opposed first and second lateral ends and perpendicular to a longitudinal axis of the second arm.

11. The instrument of claim 1, wherein the body includes a sidewall with a length extending between the proximal-facing surface and the distal-facing surface, the length being configured to limit rotation of the instrument when the instrument is disposed between opposed arms of a second surgical instrument.

12. The instrument of claim 1, further comprising at least one magnet disposed along the proximal-facing surface.

13. A surgical method, comprising:
laterally passing a portion of a first surgical instrument between opposed arms of a second surgical instrument that is coupled to an implant;
moving the first surgical instrument proximally relative to the second surgical instrument to contact a proximal-facing surface of the first surgical instrument against a distal-facing surface of the second surgical instrument;
moving a proximal end of the first surgical instrument radially outward relative to a longitudinal axis of the second surgical instrument to pivot the first surgical instrument relative to the second surgical instrument, cause a distal end of the first surgical instrument to contact an inner surface of one of the opposed arms of the second surgical instrument, and urge the arm radially outward relative to a longitudinal axis of the second surgical instrument; and
moving the first surgical instrument and the second surgical instrument proximally relative to the implant.

14. The method of claim 13, further comprising positioning the first surgical instrument relative to the second surgical instrument such that opposed first and second shoulders formed along the proximal-facing surface of the first surgical instrument abut outer surfaces of the second surgical instrument.

15. The method of claim 13, further comprising positioning the first surgical instrument such that a distal-most portion thereof is disposed at a lateral midpoint of a channel formed between the opposed arms of the second instrument.

16. The method of claim 13, further comprising coupling a modular handle to the proximal end of the first surgical instrument.

17. The method of claim 13, wherein the implant is a bone anchor.

18. The method of claim 13, wherein the second surgical instrument is a spinal rod reducer.

19. The method of claim 18, further comprising removing an inner portion of the spinal rod reducer prior to laterally passing the portion of the first surgical instrument between opposed arms of the spinal rod reducer.

20. The method of claim 13, wherein the proximal end of the first surgical instrument is part of an arm that is disposed lateral to the second surgical instrument after laterally passing the portion of the first surgical instrument between opposed arms of the second surgical instrument.

21. The method of claim 13, wherein moving the proximal end of the first surgical instrument radially outward relative to the longitudinal axis of the second surgical instrument is stopped by contact between the second surgical instrument and a portion of the first surgical instrument that is disposed between the distal end of the first surgical instrument and the proximal-facing surface of the surgical instrument.

22. The method of claim 13, wherein the opposed arms of the second surgical instrument are pivoting arms configured to latch on to a feature of the implant and the method further comprises squeezing proximal ends of the opposed arms radially inward to urge distal ends of the opposed arms radially outward while moving the proximal end of the first surgical instrument radially outward relative to the longitudinal axis of the second surgical instrument.

23. The method of claim 13, further comprising separating the first and second surgical instruments after moving the first and second surgical instruments proximally relative to the implant.
